(19) United States
(12) Patent Application Publication
(10) Pub. No.: US 2012/0165412 A1
(43) Pub. Date: Jun. 28, 2012

(54) Use of Resveratrol or Another Hydroxylated Stilbene For Preserving Cognitive Functioning

(75) Inventors: Eline Marleen van der Beek, Wageningen (NL); Mattheus Cornelis de Wilde, Elst (NL); Martine Groenendijk, Barendrecht (NL); Patrick Joseph Gerardus Hendrikus Kamphuis, Utrecht (NL)

(73) Assignee: N.V. Nutricia, Zoetermeer (NL)

(21) Appl. No.: 13/202,453

(22) PCT Filed: Feb. 19, 2010
(86) PCT No.: PCT/NL10/50085

§ 371 Date: Nov. 1, 2011

(30) Foreign Application Priority Data
Feb. 20, 2009 (NL) PCT/NL2009/050078

(51) Int. Cl. A61K 31/05; A61P 9/00; A61P 25/00; A61P 25/22; A61P 25/24; A61P 25/16; A61P 25/08; A61P 25/18; C07C 39/21; A61P 25/28

(52) U.S. Cl. 514/733; 568/729

(57) ABSTRACT

The present invention relates to the use of an hydroxylated stilbene, in particular resveratrol, in the manufacture of a neutraceutical composition for increasing the microvascular plasticity and/or microvessel density, and/or decreasing the microvessel abnormalities in the brain, in particular in the hippocampus of a mammal, in particular for the treatment of age- and condition-related decline in brain neuronal function and/or cognitive functioning in a mammal. In particular, the condition is selected from the group of Alzheimer's Disease, dementia, depression, sleep disorders, impaired memory function, psychoses, Parkinson's disease, Huntington's chorea, epilepsy, schizophrenia, paranoia, ADHD and anxiety.

### FIELD OF THE INVENTION

The present invention relates to the use of an hydroxylated stilbene in the manufacture of a neutraceutical composition for increasing the microvascular plasticity and/or microvessel density, and/or decreasing the microvessel abnormalities in the brain, and/or improving blood distribution, and/or increasing blood supply, in the brain, in particular in the hippocampus of a mammal, and/or improving nutrient supply to brain cells, and/or improving oxygen supply to brain cells and/or improving removal of metabolites from the brain tissue of a mammal.

### BACKGROUND OF THE INVENTION

Resveratrol (3,5,4′-trihydroxystilbene) is a natural polyphenol, enriched in grapes and red wine, and has a potential beneficial effect on human health. In fact, the lower incidence of cardiovascular disease in wine drinking countries with a diet high in saturated fat, a phenomenon referred to as the ‘French paradox’, is often associated with the actions of resveratrol.

Resveratrol belongs to a group of functional analogues commonly known as hydroxylated stilbenes and are further defined below. The hydroxylated stilbenes according to the invention, in particular resveratrol, can either be synthesized or isolated from natural sources, such as, for example, aqueous or ethanolic extracts of Polygonum spp. and Vitis spp.

Resveratrol has a large number of physiological effects including antioxidant, phyto-oestrogenic, vasorelaxing, anti-inflammatory and anti-carcinogenic activity. Although it is unclear which of the above effects are responsible for promoting health state, or what the underlying mechanisms are, resveratrols' ability to mimic pathways involved in caloric restriction has gained recent interest. Resveratrol can extend life span in yeast, presumably by activating the SIR2 gene, a member of the sirtuin-family, which is also implicated in the beneficial effects of caloric restriction, giving rise to a conserved deacetylase that stabilizes DNA and protects the cell. Resveratrol improves health and survival in mice on a high calorie diet, which was paralleled by an increased activity of SIRT1. Very recently, it was shown that resveratrol delays age-related deterioration and induces gene expression patterns similar to caloric restriction in normal fed mice. Another aging study shows that even a low dose of resveratrol yields a similar transcriptional pattern to that of caloric restriction, paralleled by several beneficial effects on health, although here no effects on SIRT1 were found.

Besides effects on general health and survival, a number of experimental studies suggest that resveratrol protects the brain against damage in experimental models of acute neurodegeneration as observed e.g. after stroke in ischemic brain damage models [1, 2, 3, 4, 5, 16]. Furthermore, resveratrol was shown to protect hippocampal cells against oxidative injury or stress in a rat model [17], and indicated some possible molecular sites of activity of this compound in neural and glial cells, The effect on microvessels in the brain was not investigated. Also, resveratrol has been put forward to specifically target age-related changes in cardiovascular function, in particular to have an anti-atherogenic activity) [6]. Up to now, there is no evidence if resveratrol could also protect against age-related or condition-related decline in brain function and cognitive functioning. The process of aging affects the brain in a multitude of ways and causes a progressive decline in neuronal and cognitive functioning. One of the major underlying processes in brain aging is the deterioration of the cerebrovascular system, as it is often seen in age-related and condition-related disorders such as dementia. Aging is associated with a changed microvascular plasticity, decreases in microvessel density and an increase in microvessel abnormalities such as surface irregularities. A decreased volume and efficiency in cerebral blood flow leading to a decreased nutrient and oxygen supply, may be an important first step in the development of age-related and/or condition-related cognitive impairment. Following this, the cerebrovascular system is an important target in preserving neuronal and cognitive health.

### BACKGROUND PRIOR ART PATENT DOCUMENTS

FR 2 778 337 discloses the use of monomers or polymers of polyhydroxylated stilbenes, or their corresponding glycosides, these compounds being in racemic form, or in the form of geometric isomers, for making antagonists of arylhydrocarbon receptor (AhR) ligands for treating pathologies induced by said pollutants, for the prevention and the treatment of Alzheimer's disease, associated with exposure to arylhydrocarbons. Effects on microvessels in the brain were not reported.

### SUMMARY DESCRIPTION OF THE INVENTION

The inventors examined the neurobiological effects of long term, sustained intake of a hydroxylated stilbene, an ester of a hydroxylated stilbene or an ether of a hydroxylated stilbene, in particular long term, sustained resveratrol intake in aging mammals, in particular in aging mice. To this end, in order to determine effects of long term resveratrol administration, the cerebrovascular status in the hippocampus was examined by means of electron microscopy. Additionally, an immunohistochemical analysis of the neuronal cholinergic system was performed, a system often affected in age-related cognitive decline and pathologies. To assess the effects of resveratrol on behavioural performance and cognitive function, a general explorative activity in an open field test and learning and memory in a hippocampus-dependent spatial Y-maze task was measured.

Surprisingly, the inventors found that a sustained dietary supplementation with the natural polyphenol resveratrol improved performance of aged mice in the acquisition of a Y-maze task. This improvement in cognitive performance was paralleled by an increased microvascular density in the brain, in particular the hippocampus, and a decreased number of vacuolar abnormalities in hippocampal microvessels.

In its most general embodiment, the invention is concerned with: the use of an hydroxylated stilbene, an ester of a hydroxylated stilbene or an ether of a hydroxylated stilbene, in the manufacture of a neutraceutical composition
for increasing the microvascular plasticity and/or microvessel density and/or decreasing the microvessel abnormalities in the brain, in particular in the hippocampus of a mammal;
for improving blood distribution, and/or for increasing blood supply, in the brain, in particular in the hippocampus, of a mammal; respectively
for improving nutrient supply to brain cells, and/or for improving oxygen supply to brain cells and/or for improving removal of metabolites from the brain tissue of a mammal

Hereinafter in the description and in the claims, for conciseness, the term ‘hydroxylated stilbene’ will be used to indicate the group comprising hydroxylated stilbenes, esters of hydroxylated-stilbenes and ethers of hydroxylated-stilbenes. In one aspect, the invention is concerned with the aforementioned use wherein the decline is associated with the deterioration of the cerebrovascular system, c.q. the invention is concerned with the use of an hydroxylated stilbene in the manufacture of a neutraceutical composition for the treatment or prevention of a decline in brain neuronal function and/or cognitive functioning in a mammal due to a deterioration of the cerebrovascular system.

An improved nutrient respectively oxygen supply may for instance comprise an increased supply rate and/or a more favourable distribution.

An improved removal of metabolites may for instance comprise an increased removal rate or a more effective removal.

An improved blood distribution may for instance comprised an increased distribution.

In particular a mammal having cardiovascular or cerebrovascular problems, or a mammal having an elevated risk (compared to a healthy mammal) may benefit from using a neutraceutical manufactured in accordance with the invention.

In a further aspect, the invention is concerned with the aforementioned use for the treatment or prevention of a decline in brain neuronal function and/or cognitive functioning in a mammal, c.q. the invention is concerned with the use of a hydroxylated stilbene in the manufacture of a neutraceutical composition for the treatment or prevention of a decline in brain neuronal function and/or cognitive functioning in a mammal.

In a further aspect, the invention is concerned with the aforementioned uses, wherein the decline or deterioration is age-related, i.e. is eminent in elderly mammals, and/or is condition-related, i.e. is eminent in mammals suffering from neuronal conditions such as Alzheimer's Disease (AD), dementia, depression, sleep disorders, impaired memory function, psychoses, Parkinson's disease, Huntington's chorea, epilepsy, schizophrenia, paranoia and ADHD, and for the prevention and/or treatment of anxiety. Hence, the invention is concerned with the prevention and treatment of Alzheimer's Disease, dementia, depression, sleep disorders, impaired memory function, psychoses, Parkinson's disease, Huntington's chorea, epilepsy, schizophrenia, paranoia, ADHD and anxiety. It is obvious that the decline and/or deterioration may be both age- and condition-related, such as in conditions such as age-related dementia and Alzheimer's Disease. In a specific embodiment, the neuronal condition for which the neutraceutical composition is intended is vascular dementia.

In a specific embodiment, the nutraceutical is for a subject who suffers from the metabolic syndrome, obesity, diabetes or who is insulin-resistant. Such subject may or may not have been diagnosed for a degenerative disease.

In the framework of this application, any claim directed to the use of an hydroxylated stilbene in the manufacture of a neutraceutical composition for the treatment or prevention of a condition as mentioned in this application in a mammal can be construed as a claim directed to a neutraceutical composition comprising an hydroxylated stilbene for the treatment or prevention of a condition as mentioned in this application in a mammal.

### DETAILED DESCRIPTION OF THE INVENTION

Within the context of the invention, the term “nutraceutical” as used herein, denotes the use both in the nutritional and pharmaceutical field of application. Thus, the nutraceutical composition comprising a hydroxylated stilbene can find use as supplements to food and beverages, and as pharmaceutical formulations for enteral or parenteral application which may be solid formulations such as capsules or tablets, or liquid formulations, such as solutions or suspensions. As will be evident from the foregoing, the term nutraceutical composition also comprises nutritional compositions such as, for instance bars, cookies, drinks, yogurts, ice creams, beverages and the like, containing the above-specified active ingredient.

### Hydroxylated Stilbene

The term hydroxylated stilbene as used herein, comprises compounds encompassed by the general formula I

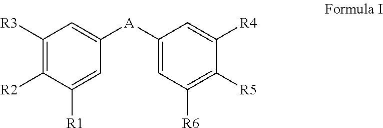

wherein A denotes a carbon-carbon double bond which may be trans or cis, and R1, R2, R3, R4, R5 and R6, independently denote hydrogen or hydroxy, with the proviso that at least one of said R-groups denotes a hydroxy group, which hydroxy group may optionally be etherified or esterified. While the carbon-carbon double bond denoted by the symbol A may be trans or cis, formula I above is understood to also include cis/trans mixtures. However, compounds of formula I wherein A is a trans carbon-carbon bond are preferred. Etherified hydroxy groups may be derived from unsubstituted or substituted, straight or branched chain alkyl groups having 1 to 26 carbon atoms, in particular 1 to 6 carbon atoms, more in particular methyl. Esterified hydroxy groups may be derived from unsubstituted or substituted, straight or branched chain aliphatic carboxylic acids having 1 to 26 carbon atoms, in particular 1 to 6 carbon atoms, more in particular formic acid, and from araliphatic and aromatic carboxylic acids having 3 to 26 carbon atoms, in particular 3 to 18 carbon atoms, more in particular 6 to 12 carbon atoms. Etherified hydroxy groups may further be glycoside groups and esterified hydroxy groups may further be glucuronide or sulphate groups. Examples of compounds of formula I are summarized in Table 1. Of primary interest for the purposes of the invention is resveratrol, in particular (trans)-resveratrol.

TABLE 1
Examples of hydroxylated stilbenes according to the invention

| | R1 | R2 | R3 | R4 | R5 | R6 |
|---|---|---|---|---|---|---|
| Resveratrol | H | OH | H | OH | H | OH |
| Pterostilbene | H | OH | H | OCH₃ | H | OCH₃ |
| Piceatannol | OH | OH | H | OH | H | OH |
| Rhapontigenin | OH | OCH₃ | H | OH | H | OH |
| Pinosylvin | H | H | H | OH | H | OH |

The nutraceutical compositions of the present invention usually contain an hydroxylated stilbene in an amount sufficient to administer to a human adult (average weight about 70 kg) a daily dosage from about 0.5 mg/day to about 2000 mg/day, preferably from about 5 mg/day to about 500 mg/day. Hence, if the nutraceutical composition is a food or beverage, the amount of a hydroxylated stilbene contained therein is suitably in the range from about 0.5 mg to about 500 mg per serving. If the nutraceutical composition is a pharmaceutical formulation, such formulation may contain from about 0.5 mg to about 500 mg per solid dosage unit, e.g., per capsule or tablet, or from about 0.5 mg per daily dose to about 2000 mg per daily dose of a liquid formulation.

The term “serving” as used herein denotes an amount of food or beverage normally ingested by a human adult with a meal at a time and may range, e.g., from about 1 g (such as a nutritional shot) to about 500 g.

In one aspect the present invention the composition comprising an hydroxylated stilbene may be used as a nutritional supplement, e.g., as an additive to a multivitamin preparations comprising vitamins and minerals which are essential for the maintenance of normal metabolic function but are not synthesized in the body, especially for the treatment or prevention of age-related decline in brain neuronal function and/or cognitive functioning in a mammal.

In a preferred embodiment, the mammal is a human. Most preferably, the human is an elderly person, except for the embodiments directed to treatment of cerebral palsy. In this respect, it is submitted that in the context of this application, an elderly person is a person of the age of 50 or more, in particular of the age of 55 or more, more in particular of the age of 60 or more, more in particular of the age of 65 or more. This rather broad definition takes into account the fact that the average age varies between different populations, on different continents, etc. Most developed world countries have accepted the chronological age of 65 years as a definition of ‘elderly’ or older person (associated with the age at which one may begin to receive pension benefits), but like many westernized concepts, this does not adapt well to e.g. the situation in Africa. At the moment, there is no United Nations (UN) standard numerical criterion, but the UN agreed cut-off is 60+ years to refer to the older population in Western world. The more traditional African definitions of an elder or ‘elderly’ person correlate with the chronological ages of 50 to 65 years, depending on the setting, the region and the country.

According to another aspect of the invention, the compositions may be pharmaceutical compositions, preferably for enteral application, which may be solid or liquid galenical formulation. Examples of solid galenical formulations are tablets, capsules (e.g. hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. While the individual active ingredients are suitably administered in a single composition they may also be administered in individual dosage units.

According to another aspect of the invention, the composition may be a nutritional composition, such as a food or beverage or a supplement composition for a food or beverage, comprising further ingredients, such as, for example, protein, fat, digestible carbohydrates, dietary fibres, such as indigestible carbohydrates, minerals, vitamins, organic acids, and flavouring agents.

According to another aspect of the invention, the composition may be a nutrition composition specifically designed for the treatment of the aforementioned conditions, comprising several further specific active ingredients, such as the ones disclosed in WO2003/041701 (N.V. Nutricia) and WO2007/073178 (N.V. Nutricia).

### Protein

Advantageously, the nutritional composition according to the invention may comprise protein, preferably intact protein. Proteins enable the manufacturing of palatable products. Especially elderly and AD patients benefit from the protein as it strengthens their motor skills. Preferably, the nutritional composition according to the invention comprises milk protein. Preferably, the nutritional composition according to the invention comprises a protein selected from the group consisting of whey protein, casein or caseinate. Preferably, the nutritional composition according to the invention comprises caseinate, more preferably the nutritional composition according to the invention comprises at least 70 weight %, more preferably at least 90 weight % casein and/or caseinate, based on total protein.

Preferably, the proteins are included in intact (unhydrolyzed) form, in order to have a palatable product. Such high molecular weight proteins increase the viscosity of the heat-treated liquid product, compared to the hydrolyzed forms. The present inventors were able to make an acceptable product, with good palatability and limited viscosity, by applying the measures according the invention, still avoiding precipitation.

Preferably, the nutritional composition according to the invention comprises between 0.2 and 16 gram protein per 100 ml, preferably between 0.2 and 10 gram protein per 100 ml, more preferably between 1 and 6 grams protein per 100 ml, more preferably between 2 and 5 grams protein per 100 ml.

### Fat

Advantageously, the nutritional composition according to the invention may comprise fat. With regard to the type of fat, a wide choice is possible, as long as the fat is of food quality.

The fat may include medium chain triglycerides (MCT, mainly 8 to 10 carbon atoms long), long chain triglycerides (LCT) or any combination of the two types.

MCTs are beneficial because they are easily absorbed and metabolized. Moreover, the use of MCTs will reduce the risk of nutrient malabsorption.

LCT sources, such as rapeseed oil, more in particular rapeseed oil low in erucic acid, sunflower oil, corn oil, palm kernel fat, coconut fat, palm oil, or mixtures thereof are preferred because they provide more energy per unit of fat.

In one embodiment, the fat is a liquid fat, i.e. an oil.

In one embodiment, the fat comprises 30 to 60 weight % of animal or algal fat, 40 to 70 weight % of vegetable fat and optionally 0 to 20 weight % of MCTs based on total fat of the nutritional composition according to the invention. The animal fat preferably comprises a low amount of milk fat, i.e. lower than 6 weight %, especially lower than 3 weight %. In particular, a mixture of corn oil, egg oil, and/or canola oil and specific amounts of marine oil are used. Egg oils, fish oils and algal oils are a preferred source of non-vegetable fats. Marine oils containing DHA and/or EPA are preferably present in the nutritional composition according to the invention in an amount lower than 25 weight %, preferably lower than 15 weight % of the fat for obtaining a maximum health effect, such as, for instance, the prevention of cardiovascular risks. The amount of EPA ranges preferably between 4 weight % and 15 weight %, more preferably between 8 weight % and 13 weight % of the fat.

Preferably, the nutritional composition according to the invention comprises a phospholipid, preferably 0.1 to 50 weight % phospholipids, based on total weight of lipids, more preferably 0.5 to 20 weight %, more preferably between 1 and 5 weight %, based on total weight of lipids. Preferably, the nutritional composition according to the invention contains at least one selected from the group consisting of phosphatidylcholine, phosphatidylethanolamine, phosphatidylserine and phosphatidylinositol. The total amount of lipids is preferably between 10 and 30 weight % on dry matter, and/or between 2 and 6 g lipid per 100 ml for a liquid composition. Inclusion of phospholipids improve the stability of the nutritional composition according to the invention.

### Digestible Carbohydrates

Advantageously, the nutritional composition according to the invention comprises digestible carbohydrates. The digestible carbohydrates positively influence the operational skills of a subject, and add to the advantageous effect of the nutritional composition according to the invention. The nutritional composition according to the invention preferably contains between 1 and 50 gram digestible carbohydrates per 100 ml of a liquid product, more preferably between 5 and 30 grams per 100 ml, more preferably 10 to 30 grams of digestible carbohydrates per 100 ml. The total amount of digestible carbohydrates is preferably between 25 and 80 weight % on dry matter basis, preferably 40 to 80 weight %.

Examples of digestible carbohydrates are digestible pentoses, digestible hexoses, and digestible oligosaccharides, e.g. digestible disaccharides and digestible trisaccharides saccharides. More specifically one or more digestible carbohydrates may be chosen selected from the group of galactose, mannose, ribose sucrose, trehalose, palatinose, lactose, maltodextrose, maltose and glucose.

### Indigestible Carbohydrates

The nutritional composition according to the invention may optionally be fortified with non-digestible carbohydrates (dietary fibres) such as oligosaccharides. These oligosaccharides may comprise fructo-oligosaccharides (FOS), galacto-oligosaccharides (GOS), trans-galacto-oligosaccharides (TOS), xylo-oligosaccharides (XOS), soy oligosaccharides, and the like. Optionally, also higher molecular weight compounds such as inulin, resistant starch and the like may be incorporated in the composition according to the invention. In another embodiment of the present invention, the composition according to the invention may comprise a mixture of neutral and acid oligosaccharides such as disclosed in WO 2005/039597 (N.V. Nutricia), the compositions therein which are incorporated herein by reference.

### Further Features

Persons suffering from neuropathies or neurological problems often experience problems with eating. Their sensory capabilities and/or control of muscles has become imparted, as well as in some instances their ambition to apply proper eating habits. Part of these patients may experience a general loss in appetite and a relatively large part of this patient group became malnourished. Preferably, the product has an energy density of 0.8 to 4.5 kcal per gram of the composition, more preferably between 0.9 and 2.5 kcal per ml.

Liquid nutritional products preferably have a long shelf life. However, increasing shelf life by heat treatments often results in destabilisation of the products and/or palatability, leading to a product which is unacceptable. The nutritional composition according to the invention can be subjected to a heat treatment without major adverse effects on the palatability. Hence, the nutritional composition according to the invention is preferably heat-treated, more preferably the composition is subjected to a sterilization treatment. In a preferred embodiment, the nutritional composition according to the invention is subjected to an ultra-high temperature treatment (UHT-treatment). Such UHT-treatment is preferably applied in line, i.e. before the liquid final product is filled in the package of the sold unit.

### EXPERIMENTAL

### A. Description of the Figures

FIG. 1. Effects of chronic resveratrol supplementation on locomotor behaviour in an open field. The test duration was 5 minutes and locomotion of the animals was monitored with an automated video analysis system. Panel A: total distance covered during the test. Panel B: time spent in the centre area of the round arena (*p=0.003).

FIG. 2. Effects of chronic resveratrol supplementation on Y-maze learning. The Y-maze task consisted of 3 training days and a retention test one week later. Each test day consisted of 8 trials and the performance of the animals is expressed as the number of correct choices per day (*p=0.016).

FIG. 3. Effects of chronic resveratrol supplementation on the cholinergic system. A. Cholinergic cell number in the nucleus basalis is unchanged in the resveratrol treated animals. B. Cholinergic fibre density in hippocampal CA1 and C. dentate gyrus does not show an effect of resveratrol treatment.

FIG. 4. Effects of chronic resveratrol supplementation on vascular density in the brain (*p=0.019). In the hippocampus, resveratrol treatment caused a higher vascular density.

FIG. 5. Effects of resveratrol supplementation on the occurrence of microvascular abnormalities. Panel A & B: a representative photomicrograph and quantitative analysis of endothelial processes. In Panel A, arrowheads are pointing at the typical endothelial processes of a capillary. Panel C & D: a representative photomicrograph and quantitative analysis of endothelial vacuoles (*p<0.05). The photomicrograph demonstrates a capillary in the hippocampus CA1 region. Arrowheads are pointing at the large, empty endothelial vacuole. Panel E & F: a representative photomicrograph and quantitative analysis of vascular basement membrane thickening. In Panel E, the photomicrograph demonstrates an arteriole in the hippocampal vascular layer. Arrowheads are pointing at healthy and thickened segments of the basement membrane. Abbreviations: asterisk: microvascular lumen, art: arterioles, cap: capillaries, e: endothelia) cell, en: endothelial cell nucleus, p: pericyte, smc: smooth muscle cell, smn: smooth muscle cell nucleus.

### METHODS

### B. Animal Testing:

### Animals and Housing

The experiment was performed with male C57BI/6 mice (Harlan, Horst, The Netherlands). Animals were individually housed under a 12 hours light/12 hours dark cycle with lights on at 8:00. Food and drinking water were provided ad libitum, except for the period of behavioural testing as described below. The experiment was started with 60 mice, half of which served as control (CON) and half of which received resveratrol in their food from the age of 1 year onwards (RES). Between the ages of 18 and 20 month, spatial learning performance of the animals was studied in a Y-maze test. At the age of 24 month, brains were collected and processed for immunocytochemistry to asses cholinergic parameters and electron microscopy for several cerebrovascular analyses. The procedures concerning animal care and treatment were in accordance with the regulations of the ethical committee for the use of experimental animals of the University of Groningen.

### Resveratrol Supplementation

Resveratrol (Hope Farms, Woerden, The Netherlands) was provided in the food (150 μg resveratrol/gram) in an amount comparable to a human dose. To rule out differences in food intake between animals on a control diet and resveratrol-supplemented diet, food intake and body weight were monitored every two weeks. To establish whether resveratrol provided in the food enters the circulation, a pilot experiment was performed in which blood was collected from mice by decapitation after 1 day and 30 days of supplementation. Plasma concentrations of resveratrol were measured. After 1 day of supplementation, resveratrol levels in the plasma were 88.5 μg/l at the end of the dark phase or active phase, when mice consume most of their food, and 41 μg/l at the end of the light phase or resting phase (n=3 each). After 1 month of supplementation, the levels were 102.7 and 30.7 μg/l at the end of active phase and rest phase, respectively (n=3 each). These measurements confirm that resveratrol ingested via the food indeed entered the circulation, and the levels appeared to fluctuate over the day in a fashion that paralleled food intake. Supplementation of resveratrol via food thus seemed an appropriate and simple method for further studies.

### Open Field Test

To examine general explorative activity, mice were subjected to an open-field test for 5 minutes. The open field consisted of a circular arena with a diameter of 120 cm. The arena was divided in two imaginary concentric zones, a central zone (60 cm diameter) and an outer zone (120 cm diameter). Position and locomotion of the mice was recorded and analyzed with a computerized video tracking system (Ethovision, Noldus Information Technology, Wageningen, The Netherlands). The number of visits and time spend in each of the two zones was determined. The open-field arena was thoroughly cleaned before a new animal was tested.

### Y Maze Learning

To examine cognitive performance, the mice were subjected to a Y-maze test in which they had to learn the location of a food reward in one of two accessible choice arms [7]. The test was conducted in a tubular, transparent Plexiglas Y maze consisting of a start arm and two test arms forming the Y. All arms of the maze were 27.5 cm long, had a diameter of 5 cm, and were at a 120° angle from each other. The home cages of the mice were equipped with a small sliding door that connected to the starting arm. One of the two test arms was baited with a food reward consisting of small crumbs of the regular food. Food crumbs were also placed below perforations at the end of the two test arms to prevent animals from discriminating between baited and non-baited arms by olfactory cues. Small grey plastic blocks (1 cm high) were placed 4 cm from the end of the arms to prevent visual inspection for food presence from a distance. A guillotine door halfway each arm could be operated manually from the experimenter's position and was used to allow animals only one choice in each training trial. The experimental room contained visual cues, which served as distal spatial cues.

To assure sufficient motivation for the test, the animals were food restricted to about 85% of their original body weight. Food restriction started 3 days before the test. The animals received their food daily between 16:00 and 18:00 PM.

The Y-maze test took place during the first half of the light phase between 10:00 and 14:00 in a room adjacent to the home cage room. The animals were carried in their home cage to the experimental room and the cage was then connected to the Y-maze. The first day of the test animals were habituated to the maze by allowing them to freely explore the arms for 5 minutes. They further received two habituation trials during which they only had access to subsequently the right or left test arm, which then contained a food reward. After the habituation day, mice received a daily training session consisting of 8 trials. During the entire training phase, either the right or left arm was baited. This was constant for a given individual, but randomized between subjects and treatments. When during a trial a subject visited one of the two accessible arms, the non-visited arm was closed. After the subject retreated to the start box, the start arm connected to the start box was blocked preventing reentrance of the maze. After cleaning all arms with damped paper cloth, and re-baiting the same arm, the subject was again allowed to explore either the right or left test arm. A visit to the baited arm was recorded as a correct trial. The mice were trained in the Y-maze for 3 consecutive days. Ten days after the final training day, animals were subjected to another series of 8 trials to test their memory.

### Tissue Processing

At the age of 24 months animals were sacrificed for histochemical and electronmicroscopical analysis. Mice were anaesthetized by a high dose of pentobarbital sodium salt (Nembutal, 60 mg/kg bodyweight) and transcardially perfused with heparinised saline followed by 4% phosphate buffered paraformaldehyde. After perfusions, brains were removed from the skull and kept in 0.01 M PBS overnight. A random subset of 18 brains were further processed for electron microscopy. Another random subset of 22 brains was used for histological analysis.

### Histology

For histology, brain tissue was cryoprotected in 30% sucrose in 0.1 M phosphate buffer for 48 h after which 20 μm sections were cut with a cryostat (Leica CM3050). To analyze possible changes in cholinergic cells and fibres, an enzymatic staining for acetylcholinesterase (AChE) was performed. Sections were postfixed in a 2.5% glutardialdehyde solution in PB overnight at 4° C. AChE histochemistry was carried out according to methods known to the skilled person. In brief, sections were incubated in an acetylthiocholine-iodide containing incubation buffer, followed by sodiumsulfide and finally intensified with a 0.1% silver nitrate solution. Stereological analysis was performed in every tenth section. Cells positive for AChE were quantified in sections containing nucleus basalis (around bregma −0.7) and AChE fibre density measurements were performed by a computerized image analysis system Quantimet (Leica) in the dorsal hippocampus.

### Electron Microscopy

After perfusion, 18 brains were prepared for electron microscopy using standard methods. Briefly, tissue blocks were incubated in an aqueous solution of 1% OsO₄ and 5% K₂Cr₄O₇ (1:1) after thorough rinsing. Subsequently, the samples were dehydrated, incubated in 1% uranyl acetate, and embedded in glycide ether. Semi-thin sections (0.3 μm) were cut on an ultramicrotome and stained on object glasses with a 1:1 mixture of 1% ethylene blue and 1% Azure II blue. The samples were then coverslipped with DPX and analyzed under a light microscope. Then, 90 nm ultra thin sections were cut of the same blocks and three non-serial sections were collected on 200 mesh copper grids. Subsequently, preparations were contrasted with 5% uranyl-acetate and Reynolds lead-citrate solution. Finally, the samples were analyzed and photographs were taken with a Philips TM10 transmission electron microscope.

For quantitative analysis, approximately 4000 μm² tissue surface was scanned systematically in each region, and 61±10 microvascular cross sections were examined in each sample. Vascular density was calculated for a standard surface area with the help of the sample grid. In addition to microvessel density, the occurrence of the following features were quantitated: luminal endothelial protrusions, large empty endothelial vacuoles and basement membrane thickening. The basement membrane was considered thickened when local exfoliations were observed in case of capillaries, or hyalinosis in case of arterioles. The number of microvessels displaying any of the noted abnormalities was counted and expressed as percentage of the total number of microvessels examined. Capillaries and arterioles were analyzed separately. Since arterioles were encountered at high enough number for statistical analysis only in the hippocampal vascular layer, quantitative analysis of arteriolar condition was performed for this region of the hippocampus alone.

### Statistical Analysis

Behavioural performance was statistically analyzed with a one way ANOVA paradigm for repeated measures. Immunocytochemical data and EM data were statistically analyzed with an independent samples T-test (2-tailed). Data are expressed as averages ±S.E.M. P<0.05 was considered as significant.

### Survival and Food Intake

Animals received resveratrol in their food from 1 year of age onwards. Adding resveratrol to the food did not affect food intake, growth and body weight (data not shown). Before behavioural testing started, at the age of 18-20 months, 4 animals died (2 CON; 2 RES). After behavioural testing, but before the end of the experiment at 24 months, 9 more animals died (4 CON; 5 RES). There were no significant differences in mortality rate and regular visual inspection did not indicate obvious differences in general health.

### Behavioural Performance

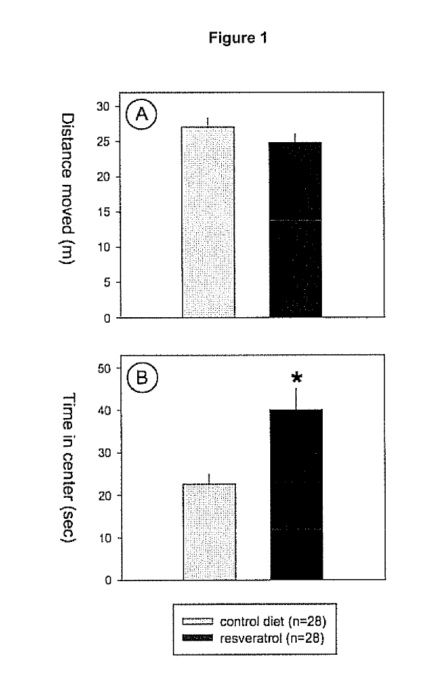

In the open field test, CON and RES mice displayed the same amount of locomotion (total distance covered), however, the RES group spent about 50% more time in the centre of the arena than the control animals (T-test, p=0.003; FIG. 1).

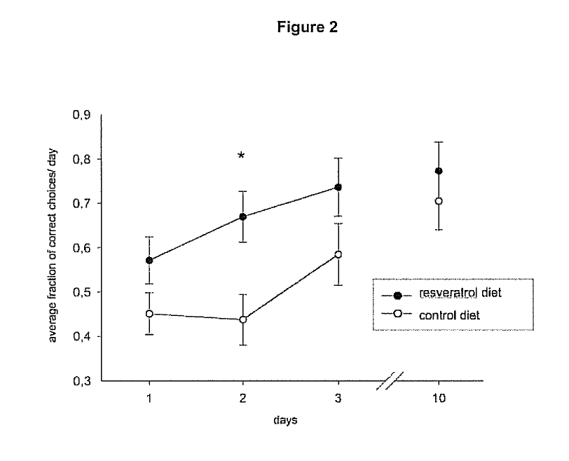

Also during the habituation trial of the Y-maze, CON and RES mice displayed similar levels of activity in terms of arm visits and spontaneous alternations (data not shown). However, during the training phase, chronic resveratrol supplementation improved learning (repeated measures ANOVA for number of correct choices on day 1 to 3: F_{1,54}=6.156, p=0.016; FIG. 2). Posthoc testing shows that RES animals performed better then CON animals on the second day of the trainings phase (p<0.05). The final level of performance on day 3 and the retention trial on day 10 were not different.

### Cholinergic Cell Numbers and Fibre Density

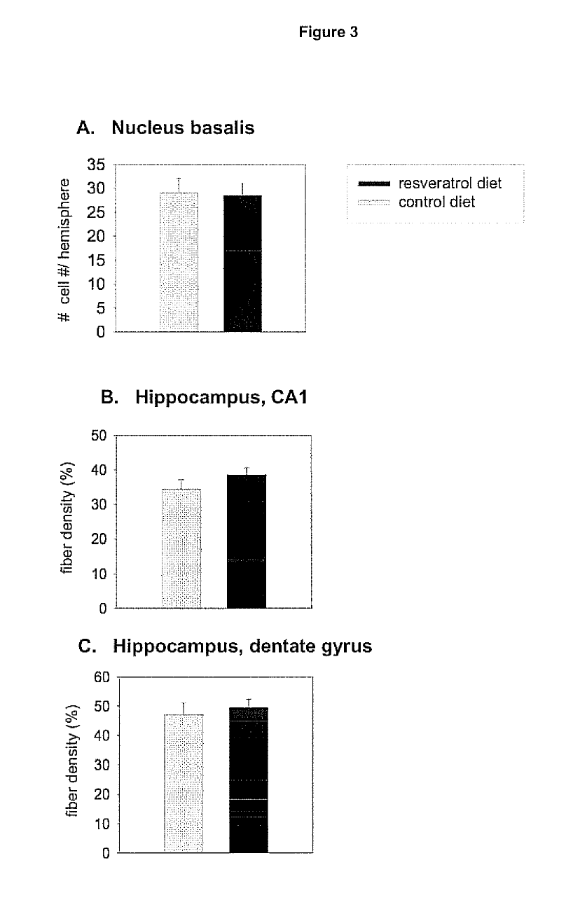

Analysis of cholinergic cell number in the nucleus basalis did not reveal any difference between CON and RES (FIG. 3A). Also, cholinergic fibre density in the CA1 and dentate gyrus of the hippocampus was unchanged after RES treatment (FIG. 3B).

### Cerebrovascular System

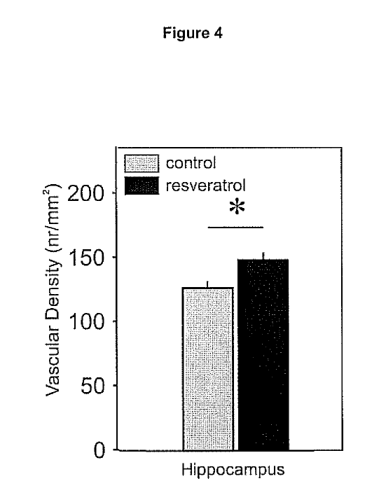

Electronmicroscopical analysis of brain tissue revealed that the vascular density in the hippocampus of RES mice was about 15% higher than that in the control animals (p=0.019; FIG. 4).

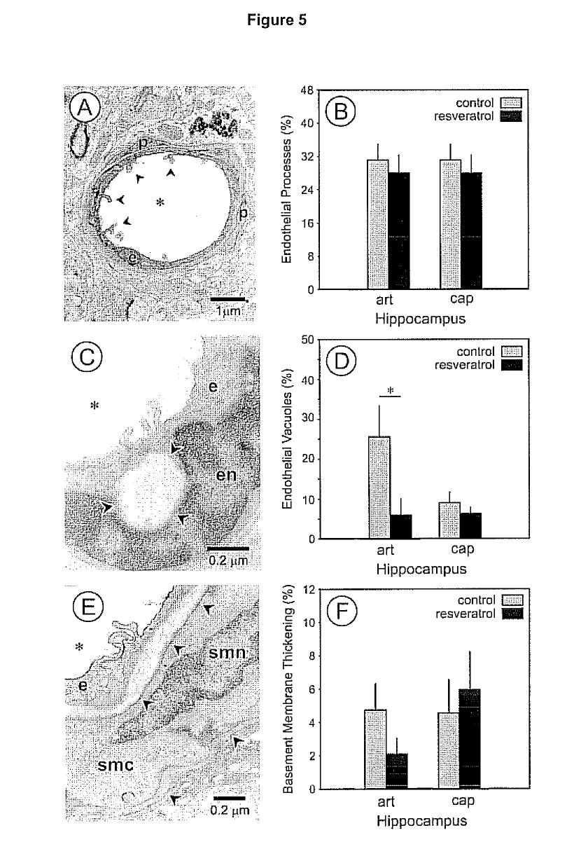

The endothelial cells of cerebral microvessels exhibited two discernable abnormalities. The apical surface of the endothelial cells displayed rich, microvillus-like processes into the lumen, and empty, large vacuoles formed preferentially in the vicinity of tight junctions (FIGS. 5A and 5C). The basement membrane of capillaries appeared occasionally thickened in the form of local exfoliations, while the arteriolar basement membrane, particularly between the endothelial and smooth muscle cells was affected by hyalinosis (FIG. 5E). The occurrence of basement membrane thickening and endothelial processes was not significantly affected by dietary resveratrol (FIGS. 5B and 5F). However, the number of microvessels with endothelial vacuoles was significantly reduced by chronic resveratrol treatment (FIG. 5D) in the hippocampal arterioles (p=0.05).

### Discussion of the Study

Chronic dietary supplementation with the natural polyphenol resveratrol improved performance of aged mice in the acquisition of a Y-maze task. This improvement in cognitive performance was paralleled by an increased microvascular density in the hippocampus and decreased number of vacuolar abnormalities in hippocampal microvessels.

Learning and memory performance in the Y-maze is dependent on the hippocampus, a brain area often affected by aging in both humans and rodents. It was found that resveratrol treated animals are better able to acquire this task. Although differences in general activity and anxiety are potential confounding factors in most learning task, it is unlikely that this explains the results of the Y-maze test in the present study. First, during the free exploration, habituation trial before the start of training, resveratrol-supplemented and control mice showed similar levels of explorative activity in terms of arm visits and spontaneous alternations. Second, during the training, all animals entered the maze with every trial and latencies were not different for treated versus untreated animals. Furthermore, the data from the open field test confirm that there was no effect of resveratrol treatment on general locomotor activity, since the total distance covered by animals was the same for both groups. All together, the increased performance of the resveratrol-treated animals during the acquisition phase of the Y-maze task most likely indicates an improved cognitive performance or, alternatively, a reduction in the well-established age-related decline in cognitive performance.

The mechanisms behind resveratrol-induced preservation of cognitive performance during aging are most likely multi-factorial. In the present study, improved performance was paralleled by higher hippocampal microvessel density and less aberrant microvessels in the hippocampus. A higher microvascular density and increased cerebral blood flow might improve performance directly by increasing glucose and oxygen supply to relevant brain areas [8]. Additionally, the microvessels potentially provide a source of trophic factors like IGF-1 and nerve growth factor (NGF) that support neuronal function. These factors have been shown to decrease in aging together with decreases in cortical microvascular density [9] and are also implicated in age-related cognitive decline [10].

Without being bound to the following explanation, an increased microvascular density can be explained in two ways. First, resveratrol could actively stimulate angiogenesis or second, resveratrol could prevent age-related decline in angiogenesis and/or deterioration of vascular status. Since resveratrol has been reported to inhibit angiogenesis in vitro [11,12], it seems more likely that resveratrol supplementation in the present study attenuated the age-related decline in vascular density and maintained normal levels of angiogenesis as opposed to an increase in angiogenesis and vessel density over control levels. Additionally, resveratrol appeared to prevent or delay the occurrence of vascular abnormalities, another hallmark of age-related decline in vascular status.

In this particular study, mice received resveratrol in a chronic fashion, doses and timing highly comparable to the experimental design. The inventors found that resveratrol produces changes associated with a longer lifespan, resulting in a physiological shift of middle-aged mice on a high-calorie diet towards that of mice on a standard diet. Survival was not assessed, but focus was on neurobiological function and cerebrovascular status. It is now postulated that one of the target-systems for resveratrol to exert its beneficial effects can be found in a preserved cerebrovascular system.

In contrast to the results on cerebrovascular level, no effects were found of chronic resveratrol treatment on structural parameters of the cholinergic system i.e. cholinergic cell number and fibre density, although direct effects of resveratrol on neuronal health cannot be ruled out. The lack of effects on neuronal level is actually paralleled by previous studies on aging, where no quantitative difference in the number of neurons or synapses were found. Furthermore, a previous studies on dietary supplementation in Tg2576 transgenic mice, a model for Alzheimer's Disease, found a strong effect on spatial learning, but no effects on neuronal readouts, like plaque deposition [13,14]. In order to exhibit its direct effects on neuronal health resveratrol is required to enter the brain in sufficient levels. In this study it was shown that resveratrol is present in the blood, fluctuating according to daily food-intake. However, the bioavailability of resveratrol and relative concentrations in different tissue are still under discussion [15] and at present, it is unknown whether sufficient concentrations of resveratrol can cross the blood-brain barrier to exert its beneficial effects directly on neuronal level. This has implications on the doses, but not on the established effect.

In conclusion, this is the first study showing beneficial effects of long-term dietary supplementation with the natural polyphenol resveratrol on cognitive functioning and cerebrovascular status. In this light, we propose that ingestion of resveratrol, thereby targeting the health of the cerebrovascular system, may be an effective and simple method to support successful aging.

### C. Example Of A Nutritional Composition

By way of example, the following liquid nutritional composition may be used as carrier for administering the hydroxylated stilbenes according to the invention, wherein the hydroxylated stilbene is resveratrol in an amount ranging from 0.2 mg to about 500 mg per serving.

Ready-to-drink oral supplement comprising per 100 ml, 100 kcal, 40 En % protein, 41.1 En % carbohydrate and 18.9 En % lipids. 10 g of protein based on whey and casein (2 g/8 g); 10.3 g carbohydrates based on maltodextrines and 2.1 g of fat based on canola oil and sunflower oil. Other micronutrients according to general recommendations for medical nutrition.

### REFERENCES

[1] Dong W, Li N, Gao D, Zhen H, Zhang X, Li F. Resveratrol attenuates ischemic brain damage in the delayed phase after stroke and induces messenger RNA and protein express for angiogenic factors. J Vasc Surg 2008.
[2] Lu K T, Chiou R Y, Chen L G, Chen M H, Tseng W T, Hsieh H T, Yang Y L. Neuroprotective effects of resveratrol on cerebral ischemia-induced neuron loss mediated by free radical scavenging and cerebral blood flow elevation. J Agric Food Chem 2006; 54(8):3126-31.
[3] Virgili M, Contestabile A. Partial neuroprotection of in vivo excitotoxic brain damage by chronic administration of the red wine antioxidant agent, trans-resveratrol in rats. Neurosci Lett 2000; 281(2-3):123-6.
[4] Wang Q, Xu J, Rottinghaus G E, Simonyi A, Lubahn D, Sun G Y, Sun A Y. Resveratrol protects against global cerebral ischemic injury in gerbils. Brain Res 2002; 958(2):439-47.
[5] Zhang H, Schools G P, Lei T, Wang W, Kimelberg H K, Zhou M. Resveratrol attenuates early pyramidal neuron excitability impairment and death in acute rat hippocampal slices caused by oxygen-glucose deprivation. Exp Neurol 2008; 212(1):44-52.
[6] Labinskyy N, Csiszar A, Veress G, Stef G, Pacher P, Oroszi G, Wu J, Ungvari Z. Vascular dysfunction in aging: potential effects of resveratrol, an anti-inflammatory phytoestrogen. Curr Med Chem 2006; 13(9):989-96.
[7] Havekes R, Timmer M, Van der Zee E A. Regional differences in hippocampal PKA immunoreactivity after training and reversal training in a spatial Y-maze task. Hippocampus 2007; 17(5):338-48.

[8] Gold P E. Glucose and age-related changes in memory. Neurobiol Aging 2005; 26 Suppl 1:60-4.
[9] Sonntag W E, Lynch C D, Cooney P T, Hutchins P M. Decreases in cerebral microvasculature with age are associated with the decline in growth hormone and insulin-like growth factor 1. Endocrinology 1997; 138(8):3515-20.
[10] Ramsey M M, Weiner J L, Moore T P, Carter C S, Sonntag W E. Growth hormone treatment attenuates age-related changes in hippocampal short-term plasticity and spatial learning. Neuroscience 2004; 129(1):119-27.
[11] Cao Z, Fang J, Xia C, Shi X, Jiang B H. trans-3,4,5′-Trihydroxystibene inhibits hypoxia-inducible factor 1 alpha and vascular endothelial growth factor expression in human ovarian cancer cells. Clin Cancer Res 2004; 10(15):5253-63.
[12] Igura K, Ohta T, Kuroda Y, Kaji K. Resveratrol and quercetin inhibit angiogenesis in vitro. Cancer Lett 2001; 171(1):11-6.
[13] Quinn J F, Bussiere J R, Hammond R S, Montine T J, Henson E, Jones R E, Stackman R W, Jr. Chronic dietary alpha-lipoic acid reduces deficits in hippocampal memory of aged Tg2576 mice. Neurobiol Aging 2007; 28(2):213-25.
[14] Stackman R W, Eckenstein F, Frei B, Kulhanek D, Nowlin J, Quinn J F. Prevention of age-related spatial memory deficits in a transgenic mouse model of Alzheimer's disease by chronic Ginkgo biloba treatment. Exp Neurol 2003; 184(1):510-20.
[15] Rossi L, Mazzitelli S, Arciello M, Capo C R, Rotilio G. Benefits from Dietary Polyphenols for Brain Aging and Alzheimer's Disease. Neurochem Res 2008.
[16] Raval A. P., Hung Wen Lin, Kunjan R. D., Defazio R. A., Della Morte D., Eun Joo Kim en Perez-Pinzon M. A. Resveratrol and ischaemic preconditioning in the brain, Current Medicinal Chemistry, 2008, 15, 1545-1551.
[17] Lucia Maria Vieira de Almeida et al. Resveratrol protects against oxidative injury induced by H₂O₂ in acute hippocampal slice preparations from Wistar rats. Archives of Biochemistry and Biophysics 480 (2008) 27-32.

1. Use of an hydroxylated stilbene in the manufacture of a neutraceutical composition for increasing the microvascular plasticity and/or for increasing microvessel density and/or for decreasing the microvessel abnormalities, in the brain, in particular in the hippocampus, of a mammal.

2. Use of an hydroxylated stilbene in the manufacture of a neutraceutical composition for improving blood distribution, and/or for increasing blood supply, in the brain, in particular in the hippocampus, of a mammal.

3. Use of an hydroxylated stilbene in the manufacture of a neutraceutical composition for improving nutrient supply to brain cells, and/or for improving oxygen supply to brain cells and/or for improving removal of metabolites from the brain tissue of a mammal.

4. Use of an hydroxylated stilbene, optionally according to any one of the claims 1-3, in the manufacture of a nutraceutical composition for the treatment of vascular dementia.

5. Use according to any one of the claims 1 to 4, for the treatment or prevention of a decline in brain neuronal function and/or cognitive functioning in a mammal.

6. Use according claim 5, wherein the decline is associated with the deterioration of the cerebrovascular system.

7. Use according to claim 5 or 6, wherein the decline is age-related and/or condition-related.

8. Use according to any one of claims 1 to 7 wherein the nutraceutical composition is for the treatment of a mammal suffering from a condition selected from the group of Alzheimer's Disease, dementia, depression, sleep disorders, impaired memory function, psychoses, Parkinson's disease, Huntington's chorea, epilepsy, schizophrenia, paranoia, ADHD and anxiety.

9. Use according to any one of the preceding claims, wherein a subject is treated selected from the group of subjects suffering from a transient ischemic accident, babies suffering from cerebral palsy, subjects suffering from metabolic syndrome, obese subjects, diabetics, and insulin-resistant subjects.

10. Use according to any one of the preceding claims, wherein the mammal is a human, preferably an elderly human.

11. Use of a composition according to any one of the preceding claims wherein the hydroxylated stilbene is present in an amount sufficient to administer in a daily dosage of about 0.5 mg/day to about 2000 mg/day.

12. Use of a composition according to any one of the preceding claims, wherein the composition is in dosage unit form.

13. Use of a composition according to claim 12, wherein the dosage unit form is a solid dosage unit form.

14. Use of a composition according to claim 12 or 13, wherein the dosage unit form contains about 0.5 mg to about 500 mg of hydroxylated stilbene.

15. Use of a composition according to any one of claims 1 to 11, wherein the nutritional composition is a food or beverage or a supplement composition for a food or beverage.

16. Use of a composition according to claim 15, wherein the hydroxylated stilbene is present in an amount of about 0.5 mg to about 500 mg.

17. Use of a composition according to any one of claim 15 or 16, further comprising at least one component selected from the group of fat, proteins and carbohydrates.

18. Use of a composition according to any one of the preceding claims, wherein the hydroxylated stilbene is selected from the group of resveratrol, pterostilbene, piceatannol, rhapontigenin and pinosylvin.

19. Use according to claim 18, wherein the hydroxylated stilbene is resveratrol.
